(19) United States
(12) Patent Application Publication
(10) Pub. No.: US 2009/0124647 A1
(43) Pub. Date: May 14, 2009

(54) Indole Derivatives as Antitumoral Compounds

(75) Inventors: Jose Fernando Reyes Benitez, Madrid (ES); Andres Francesch Solloso, Madrid (ES); Carmen Cuevas Marchante, Madrid (ES); Marta Altuna Urquijo, Barcelona (ES); Daniel Pla Queral, Barcelona (ES); Mercedes Alvarez Domingo, Barcelona (ES); Fernando Albericio Palomera, Barcelona (ES)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 12/093,563

(22) PCT Filed: Nov. 13, 2006
(86) PCT No.: PCT/GB06/50386

§ 371 Date: Jul. 21, 2008

(30) Foreign Application Priority Data
Nov. 14, 2005 (GB) 0523098.2

(51) Int. Cl. A61K 31/506; C07D 403/04

(52) U.S. Cl. 514/275; 544/297

(57) ABSTRACT

Antitumoural compounds of general formula (I); wherein Ar is an heterocyclic group of formula (a) and R₁, R₂, R₃, R4, R₅, R₆, R₇, n and the dotted line take permitted meanings can be obtained from a tunicate of the family Polyclinidae, genus Aplidium, species cyaneum, and the invention further provides derivatives thereof.

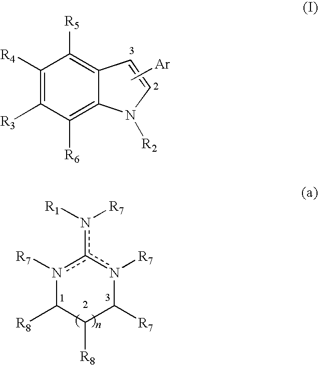

### FIELD OF THE INVENTION

The present invention relates to new antitumoural compounds, pharmaceutical compositions containing them and their use as antitumoural agents.

### BACKGROUND OF THE INVENTION

Several indole alkaloids have been disclosed to have cytotoxic properties against tumor cell lines. See for example Hernández Franco L. et al (J. Nat. Prod., 1998, 61, 1130-1132), which discloses meridianins A-E isolated from the tunicate Aplidium meridianum, showing cytotoxicity toward LMM3 cell line with IC₅₀ values between 9.3 μM and 33.9 μM.

In addition, several meridianins have been disclosed as inhibitors of various protein kinases such as cyclin-dependent kinases, glycogen synthase kinase-3, cyclic nucleotide-dependent kinase and casein kinase 1 (Gompel M. et al. Bioorganic & Medicinal Chemistry Letters, 2004, 14,1703-1707).

Several indolylpyrimidines and indolylpyrazines have been also disclosed as potential antitumor agents by Jiang B. et al (Bioorganic & Medicinal Chemistry, 2001, 9, 1149-1154). It is disclosed that 2,3-bis(3-indolyl)pyrimidine (compound 8) displayed strong selective cytotoxic activity against IGROV1 tumor cell line with GI₅₀ values below 0.01 μM and 2-amino-3methoxyl-5-(3′-indolyl)pyrazine (compound 19) exhibited excellent selective inhibition against CCRF-CEM cancer lines (GI₅₀=0.81 μM) and HOP-92 cancer lines (GI₅₀=0.03 μM).

Finally, two aminoimidazolinyl indole compounds have been disclosed to inhibit the in vitro proliferation of different types of cancer cells. Specifically, Sun H. H. and Sakemi S. (J. Org. Chem., 1991, 56, 4307-4308) discloses that discodermindole yielded IC₅₀ values of 1.8 μg/mL against P388, 4.6 μg/mL against A-549, and 12 μg/mL against HT-29 cell lines. On the other hand, Cohen J. et al. (Pharmaceutical Biology, 2004, 42(1), 59-61) discloses that 6-hydroxydiscodermindole inhibited the in vitro proliferation of cultured P388 and A-549 cells with IC₅₀ values of 4.6 and >5 μg/mL, respectively. In addition, this paper discloses the hydrogenolysis of 6-hydroxydiscodermindole giving a compound with the following structure:

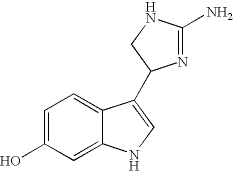

No activity data is reported for this compound.

Cancer is a leading cause of death in animals and humans. Huge efforts have been and are still being undertaken in order to obtain an antitumor agent active and safe to be administered to patients suffering from a cancer. The problem to be solved by the present invention is to provide compounds that are useful in the treatment of cancer.

### SUMMARY OF THE INVENTION

In one aspect, the present invention is directed to antitumor compounds of general formula I or a pharmaceutically acceptable salt, derivative, tautomer, prodrug or stereoisomer thereof

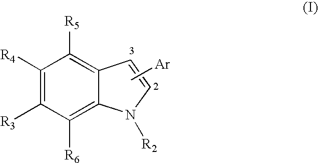

wherein Ar is an heterocyclic group of formula

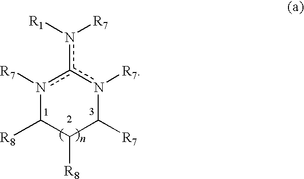

Bach R₁, R₂ and R₇ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, NRₐR_{b}, NRₐCOR_{b}, SO₂Rₐ, COORₐ, CORₐ, CONRₐR_{b}, ORₐ and OCORₐ.

Each R₃, R₄, R₅, R₆ and R₈ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted aryalkyl, substituted or unsubstituted arylalkenyl, substituted or unsubstituted heterocyclic group, halogen, CN, NO₂, COORₐ, CORₐ, CONRₐR_{b}, ORₐ, OCORₐ, NRₐR_{b} and NRₐCOR_{b}.

n is selected from 0 and 1.

Each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, and substituted or unsubstituted heterocyclic group.

The Ar group may be attached to the carbon atoms 2 or 3 of the indole group through its atoms 1, 2 or 3.

The dotted lines represent one optional additional bond, with the proviso that when said additional bond exists the N atom bearing the double bond lacks the R₇ group.

In another aspect, the present invention is directed to pharmaceutical compositions comprising a compound of formula I, as defined above, or a pharmaceutically acceptable salt, derivative, tautomer, prodrug or stereoisomer thereof together with a pharmaceutically acceptable carrier or diluent.

In another aspect, the present invention is also directed to the use of compounds of formula I, as defined above, or a pharmaceutically acceptable salt, derivative, tautomer, prodrug or stereoisomer thereof in the treatment of cancer, or in the preparation of a medicament for the treatment of cancer. Other aspects of the invention arc methods of treatment, and compounds for use in these methods. Therefore, the present invention further provides a method of treating any mammal, notably a human, affected by cancer which comprises administering to the affected individual a therapeutically effective amount of a compound as defined above.

The present invention also relates to the isolation of the compounds of formula I from a tunicate of the family Polyclinidae, genus Aplidium, species cyaneum, and the formation of derivatives from these compounds.

### DETAILED DESCRIPTION OF PREFERRED EMBODIMENTS

The present invention relates to compounds of general formula I as defined above.

In these compounds the substituents can be selected in accordance with the following guidance:

Alkyl and alkoxy groups may be branched or unbranched and preferably have from 1 to 12 carbon atoms. One more preferred class of alkyl and alkoxy groups has from 1 to about 6 carbon atoms. Methyl, ethyl, propyl, butyl and pentyl including isopropyl, isobutyl and isopentyl are particularly preferred alkyl groups in the compounds of the present invention. Methoxy, ethoxy, propoxy including isopropoxy are particularly preferred alkoxy groups in the compounds of the present invention.

Preferred alkenyl and alkynyl groups in the compounds of the present invention have one or more unsaturated linkages, may be branched or unbranched and have from 2 to about 12 carbon atoms. One more preferred class of alkenyl groups has from 2 to about 6 carbon atoms. One more preferred class alkynyl groups has from 2 to about 6 carbon atoms.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Specially preferred aryl groups include substituted or unsubstituted phenyl, naphthyl, biphenyl, phenanthryl and anthracyl.

Suitable heterocyclic groups include heteroaromatic and heteroalicyclic groups. Suitable heteroaromatic groups in the compounds of the present invention contain one, two or three heteroatoms selected from N, O or S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, imidazolyl, indolyl, benzofuranyl and benzothiazol groups. Suitable heteroalicyclic groups in the compounds of the present invention contain one, two or three heteroatoms selected from N, O or S atoms and include, e.g., tetrahydrofuranyl, tetrahydropyranyl, piperidinyl, morpholino and pyrrolindinyl groups.

Preferred arylalkyl and arylalkenyl groups are those in which the alkylchain and the alkenylchain may be branched or unbranched and preferably have from 1 to 12 carbon atoms and from 2 to 12 carbon atoms, respectively. One more preferred class of alkylchain has from 1 to about 6 carbon atoms, and one more preferred class of alkenylchain has from 2 to about 6 carbon atoms. Preferred aryl moieties in the arylalkyl and arylalkenyl groups include single and multiple ring moieties, including multiple ring moieties that contain separate and/or fused aryl groups. Typical aryl moieties contain from 1 to 3 separated or fused rings and from 6 to about 18 carbon ring atoms. Specially preferred aryl moieties include substituted or unsubstituted phenyl, naphthyl, biphenyl, phenanthryl and anthracyl. Therefore, suitable arylalkyl and arylalkenyl groups in the compounds of the present invention have from 7 to 30 carbon atoms and from 8 to 30 carbon atoms, respectively.

The groups above mentioned may be substituted at one or more available positions by one or more suitable groups such as OR′, =O, SR′, SOR′, SO₂R′, NO₂, NHR′, N(R′)₂, =N—R′, NHCOR′, N(COR′)₂, NHSO₂R′, CN, halogen, COR′, CO₂R′, OCOR′, CONHR′, CON(R′)₂, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl and substituted or unsubstituted heterocyclic group, wherein each of the R′ groups is independently selected from the group consisting of H, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl and substituted or unsubstituted heterocyclic group. Suitable halogen substituents in the compounds of the present invention include F, Cl, Br and I. Where such groups are themselves substituted, the substituents may be chosen from the foregoing list.

The term “pharmaceutically acceptable salts, derivatives, prodrugs” refers to any pharmaceutically acceptable salt, ester, solvate, hydrate or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline form either as free compounds or as solvates (e.g. hydrates) and it is intended that both forms are within the scope of the present invention. Methods of solvation are generally known within the art.

Any compound that is a prodrug of a compound of formula I is within the scope and spirit of the invention. The term “prodrug” is used in its broadest sense and encompasses those derivatives that are converted in vivo to the compounds of the invention. Such derivatives would readily occur to those skilled in the art, and include, for example, compounds where a free hydroxy group is converted into an ester derivative.

Any compound referred to herein is intended to represent such specific compound as well as certain variations or forms. In particular, compounds referred to herein may have asymmetric centres and therefore exist in different enantiomeric forms. All optical isomers and stercoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof.

Furthermore, compounds referred to herein may exist as geometric isomers (i.e., cis and trans isomers), as tautomers, or as atropisomers. Specifically, the term tautomer refers to one of two or more structural isomers of a compound, that exist in equilibrium and are readily converted from one isomeric form to another. Common tautomeric pairs are amine-imine, amide-imide, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Preferred compounds of the invention are those of general formula II

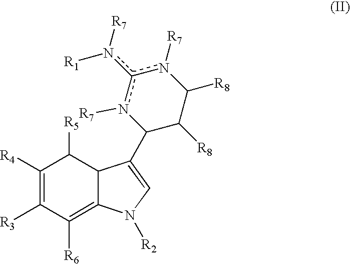

wherein R₁-R₈ groups have the same meaning given above.

Particularly preferred compounds are those wherein R₁ and R₇ are independently selected from hydrogen, substituted or unsubstituted C₁-C₁₂ alkyl, substituted or unsubstituted aryl, ORₐ and CORₐ, and wherein Rₐ has the same meaning given above.

Particularly preferred R₂ is hydrogen, substituted or unsubstituted C₁-C₁₂ alkyl, substituted or unsubstituted aryl, ORₐ and CORₐ, and wherein Rₐ has the same meaning given above.

Particularly preferred R₃, R₄, R₅ and R₆ are hydrogen, halogen, OR₁, OCORₐ, NRₐR_{b}, NRₐCOR_{b}; and wherein Rₐ and R_{b} have the same meaning given above.

Particularly preferred R₈ is hydrogen, halogen, NRₐR_{b} and NO₂, and wherein Rₐ and R_{b} have the same meaning given above.

In addition, the presence of one additional bond in one of the dotted lines is preferred.

Furthermore, particularly preferred compounds of the present invention are those of general formula III

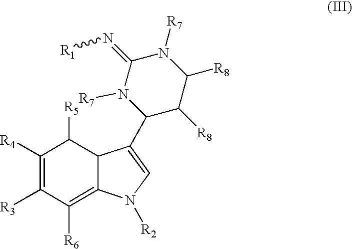

wherein R₁ is preferably selected from hydrogen and CORₐ, wherein Rₐ is a substituted or unsubstituted C1-C6 alkyl, being mcthyl the most preferred;

R₂ is preferably hydrogen and ORₐ, wherein Rₐ is a substituted or unsubstituted C1-C6 alkyl, being methyl the most preferred;

R₃, R₄, R₅ and R₆ are preferably independently selected from hydrogen and halogen, being Br the preferred halogen;

R₇ and R₈ are preferably hydrogen; and

the wavy bond ( [symbol] ) means that the double bond can exist as (E)-isomer or (Z)-isomer.

Particularly preferred compounds of the invention are the following:

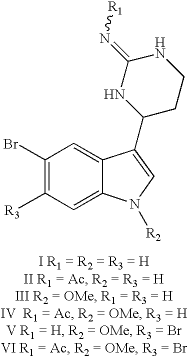

Compounds of the invention are readily made by synthetic methods. For example, compounds of this invention can be obtained adapting the procedures described in Fresneda P. et al. Tetrahedron Letters, 2000, 41, 4777-4780; Fresneda P. et al. Tetrahedron, 2001, 57, 2355-2363; and Jiang B. et al. Bioorganic & Medicinal Chemistry, 2001, 9, 1149-1154. The synthetic routes can use combinations of steps taken from more than one of these articles.

For example, compounds of the invention can be made following the synthetic sequence indicated in the Scheme 1.

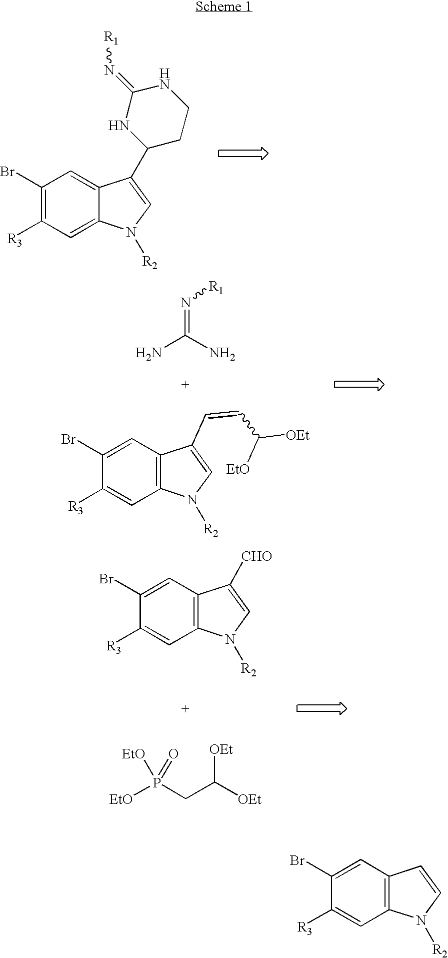

wherein R₁, R₂ and R₃ are the desired substituents and arc as defined above.

This process can comprise the following key steps:

a) Formylation of the corresponding substituted indole by a Vilsmeier-Haack reaction to afford the corresponding aldehyde. For this reaction the procedures disclosed in Hanley A B. et al. (J. Chem. Soc. Perkin Trans. 1. 1990, 2273-2276) can be used.

b) Horner-Wadsworth-Emmons reaction between the aldehyde previously obtained and diethyl 2,2-diethoxyethylphosphonate to obtain the protected substituted acryaldehyde. For this reaction the procedures disclosed in Mouloungui Z. et al. (Syn. Comm. 1988, 18, 1241-1245) can be used.

c) Formation of the tetrahydropyrimidin-2(1H)-imine ring can be done by direct treatment of the substituted acryaldehyde with guanidine following procedures described in the literature (Weis A L. and Zamir D J. J. Org. Chem. 1987, 52, 3421-3425) to afford the desired compounds.

Analogues can be synthesized by an equivalent process as those described, by choosing the appropriate substituents of the intermediate compounds in each case.

When necessary, appropriate protecting groups can be used on the substituents to ensure that reactive groups are not affected. The synthesis can be designed to employ precursor substituents, which can be converted at the appropriate stage to a desired substituent. Saturation or unsaturation in the ring-structure can be introduced or removed as part of the synthesis. Starting materials and reagents can be modified as desired to ensure synthesis of the intended compound. In addition, analogues can also be synthesized by usual procedures in synthetic organic chemistry and already known by a person skilled in the art.

In addition, some of the compounds of this invention can be of marine origin.

Compounds I-VI were isolated from a tunicate, of the family Polyclinidae, genus Aplidium, species cyaneum. Two samples of the specimen were deposited in the Department of Environmental Sciences (Marine Biology Unit) of the University of Alicante (Spain) with the following reference codes: ASC.ANT.EQ.433-1 and ASC.ANT.EQ.1097-1. This tunicate was collected by bottom trawling in Weddell Sea (Longitude: −10.533333, Latitude: −71.933333) at a depth ranging between 220 and 300 m, and its description is the following:

Aplidium cyaneum, also known as Aplidium caeruleum, is distributed at the circum-Antarctic in waters of the continental shelf and slope from 75 down to about 1000 meters. Colonies are usually upright, club-shaped. There may be 2 lobes or heads from a single base, or in particularly wide colonies there may be 2 inverted cone-shaped bases supporting a widely spreading upper part of the colony. Height of colonies generally about 4 cm. The outer layer of test is skin-like and tough, or quite brittle with sand. Internally the test is soft and in preserved specimens is pigmented bright blue or red. The pigmentation is not always evenly distributed and is often confined to blood vessels and membranous fibers in the test, while the matrix of the test is colourless. Zooids are arranged in the test in circular systems of 6 to 15 zooids around a conspicuous central common cloaca. Zooids are large, often as much as 12 mm long in the contracted state and up to 3 mm wide in the thoracic region. The branchial aperture has 6 lobes. The atrial aperture, with well developed sphincter muscle, is often produced almost into a siphon with a stout languet, of varying length, sometimes divided into 3 or even 4 lobes from the anterior border of the opening. There is a narrow frilled atrial velum in the base of the atrial siphon. The musculature is well developed with about 20 longitudinal bands on the thorax extending along both sides of the ventral aspect of the abdomen and posterior abdomen. The branchial sac is wide with 6 to 20 rows of stigmata, often all with parastigmatic vessels, with parastigmatic vessels absent from the most posterior rows, or with no parastigmatic vessels. The esophagus is narrow and the stomach rather ‘shield-shaped’ with 10 to 13 shallows folds, often broken and irregular, especially on the side against the intestine. Folds may be completely absent. The anal border is fringed by about 12 long finger-like lobes. The posterior abdomen is often long and sturdy with the testis follicles multilobed and in double rows with a clump of ova anteriorly.

An important feature of the above described compounds of formula I is their bioactivity and in particular their cytotoxic and antimitotic activity.

With this invention we provide novel pharmaceutical compositions of compounds of general formula I that possess cytotoxic and antimitotic activity, and their use as antitumor agents. Thus the present invention further provides pharmaceutical compositions comprising a compound of this invention, or a pharmaceutically acceptable salt, derivative, prodrug or stereoisomer thereof with a pharmaceutically acceptable carrier.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The compounds and compositions of this invention may be used with other drugs to provide a combination therapy. The other drugs may form part of the same composition, or be provided as a separate composition for administration at the same time or at different time.

Antitumoural activities of these compounds include, but are not limited, activity against lung cancer, colon cancer, breast cancer and cervix cancer.

### EXAMPLES

### Example 1

### Description of the Marine Organism and Collection Side

Aplidium cyaneum was collected by bottom trawling in Weddell Sea (Longitude: −10.533333, Latitude: −71.933333) at a depth ranging between 220 and 300 m. Two samples of the specimen were deposited in the Department of Environmental Sciences (Marine Biology Unit) of the University of Alicante (Spain). Their reference codes are ASC.ANT.EQ.433-1 and ASC.ANT.EQ. 1097-1.

### Example 2

### Isolation of Compounds I-VI

The frozen organism (437 g) was diced and extracted with H₂O (1 L+2×300 mL) and a mixture of MeOH:CH₂Cl₂ (1:1) (3×500 mL) at room temperature. The organic extract was evaporated under reduced pressure to yield a crude of 939.7 mg. This material was chromatographed (VLC) on Lichroprep RP-18 with a stepped gradient from H₂O to MeOH and subsequently MeOH:CH₂Cl₂ (1:1) and CH₂Cl₂. The fraction eluted with H₂O: MeOH (1:1, 51.3 mg) was subjected to semipreparative reversed phase HPLC (SymmetryPrep C18, 7.8×150 mm, 7 μm, gradient H₂O+0.1% TFA:CH₃CN+0.1% TFA, from 10 to 60% CH₃CN+0.1% TFA in 20 min, flow 2.3 mL/min, UV detection at 254 nm) to yield Compound I (1.6 mg) in the form of its trifluoroacetate salt, Compound II (5.1 mg), Compound III (5.3 mg) in the form of its trifluoroacetate salt, Compound IV (14.7 mg), Compound V (10.5 mg) in the form of its trifluoroacetate salt and Compound VI (11.4 mg).

Compound I: pale yellow oil. [α]²⁵_{D}−0.8° (c 0.1, CHCl₃); IR (NaCl) vₘₐₓ 3369, 2922, 1668, 1627, 1459, 1198, 1134 cm⁻¹; (+)-HRESIMS m/z 293.0399 [M+H]⁺(Calcd. for C₁₂H₁₄N₄⁷⁹Br 293.0396). ¹H (500 MHz) and ¹³C NMR (125 MHz) see Table 1.
Compound II: pale yellow oil. [60 ]²⁵_{D}+8.7° (c 0.1, CHCl₃); IR (NaCl) vₘₐₓ 3430, 1661, 1436, 1257, 1200, 1138 cm⁻¹; (+)-HRESIMS m/z 335.0508 [M+H]⁺(Calcd. for C₁₄H16N₄⁷⁹BrO 335.0501); ¹H (500 MHz) and ¹³C NMR
Compound III: pale yellow oil. [α]²⁵_{D}+3.1° (c 0.1, CHCl₃); IR (NaCl) vₘₐₓ 3373, 1668, 1627, 1438, 1201, 1137 cm⁻¹; (+)-HRESIMS m/z 323.0516 [M+H]⁺(Calcd. for C₁₃H₁₆N₄⁷⁹BrO 323.0501). ¹H (500 MHz) and ¹³C NMR (125 MHz) see Table 2.
Compound IV: pale yellow oil. [α]²⁵_{D}+9.5° (c 0.1, CHCl₃); IR (NaCl) vₘₐₓ 3433, 1670, 1451, 1259, 1200, 1134, cm⁻¹; (+)-HRESIMS m/z 365.0611 [M+H]⁺(Calcd. for C₁₅H₁₈N₄⁷⁹BrO₂ 365.0607). ¹H (500 MHz) and ¹³C NMR (125 MHz) see Table 2.
Compound V: pale yellow oil. [α]²⁵_{D}+0.5° (c 0.1, CHCl₃); IR (NaCl) vₘₐₓ 3411, 1672, 1439, 1201, 1135, cm⁻¹; (+)-HRESIMS m/z 400.9609 [M+H]⁺(Calcd. for C₁₃H₁₅N₄⁷⁹Br₂O 400.9607). ¹H (500 MHz) and ¹³C NMR (125 MHz) see Table 3.
Compound VI: pale yellow oil. [α]²⁵_{D}+1.9° (c 0.1, CHCl₃); IR (NaCl) vₘₐₓ 3440, 1680, 1440, 1260, 1201, 1135, cm⁻¹; (+)-HRESIMS m/z 442.9734 [M+H]⁺(Calcd. for C₁₅H₁₇N₄⁷⁹Br₂O 442.9712). ¹H (500 MHz) and ¹³C NMR (125 MHz) see Table 3.

TABLE 1
¹H and ¹³C NMR data of Compound I and II (CD₃OD, 500 and
125 MHz).

| | Compound I | | Compound II | |
|---|---|---|---|---|
| N° | ¹H (Multiplicity, J) | ¹³C | ¹H (Multiplicity, J) | P¹³C |
| 2 | 7.31 (s) | 125.3 | 7.37 (s) | 125.6 |
| 3 | — | 113.6 | — | 113.8 |
| 4 | — | 124.1 | — | 127.8 |
| 5 | 7.75 (d, 2.0) | 121.9 | 7.80 (d, 2.0) | 121.8 |
| 6 | | 115.2 | — | 114.3 |
| 7 | 7.26 (dd, 9.0, 2.0) | 125.9 | 7.29 (dd, 8.5, 2.0) | 126.1 |
| 8 | 7.34 (d, 9.0) | 114.5 | 7.36 (d, 8.5) | 114.6 |
| 9 | | 137.2 | — | 137.2 |
| 10 | 4.95 (dd, 6.5, 6.0) | 48.1 | 5.14 (dd, 6.5, 6.0) | 48.2 |
| 11 | 2.25 (m), 2H | 28.3 | 2.34 (m), 2H | 26.9 |
| 12 | 3.46 (ddd, 12.5, 6.5, 6.5) | 38.6 | 3.60 (ddd, 13.0, 7.5, | 38.6 |
| | 3.41 (ddd, 12.5, 5.0, 5.0) | | 6.5) | |
| | | | 3.53 (ddd, 13.0, 5.5, | |
| | | | 5.0) | |
| 13 | — | 155.7 | — | 152.3 |
| OCH₃ | — | — | — | — |
| CH₃CO | — | — | — | 173.9 |
| CH₃CO | — | — | 2.21 (s) | 24.1 |

TABLE 2
¹H and ¹³C NMR data of Compound III and IV (CD₃OD, 500 and
125 MHz).

| | Compound III | | Compound IV | |
|---|---|---|---|---|
| N° | ¹H (Multiplicity, J) | ¹³C | ¹H (Multiplicity, J) | ¹³C |
| 2 | 7.56 (s) | 123.8 | 7.61 (s) | 124.1 |
| 3 | — | 112.0 | — | 111.2 |
| 4 | — | 124.3 | — | 124.1 |
| 5 | 7.80 (d, 2.0) | 122.5 | 7.83 (d, 1.5) | 122.4 |
| 6 | — | 114.4 | — | 114.6 |
| 7 | 7.37 (dd, 8.5, 2.0) | 126.9 | 7.38 (dd, 8.5, 1.5) | 127.1 |
| 8 | 7.42 (d, 8.5) | 111.4 | 7.42 (d, 8.5) | 111.5 |
| 9 | — | 132.7 | — | 132.7 |
| 10 | 4.94 (dd, 8.0, 4.5) | 47.6 | 5.11 (dd, 7.5, 5.0) | 47.7 |
| 11 | 2.26 (dddd, 13.5, 8.5, 8.0, | 28.3 | 2.32 (dddd, 13.5, | 26.6 |
| | 5.5) | | 8.0, 7.5, 6.5) | |
| | 2.18 (dddd, 13.5, 5.5, 4.5, | | 2.18 (dddd, 13.0, | |
| | 4.5) | | 5.0, 5.0, 4.5) | |
| 12 | 3.45 (ddd, 12.5, 8.5, 4.5) | 38.4 | 3.57 (ddd, 13.5, | 38.2 |
| | 3.40 (ddd, 12.5, 5.5, 5.0) | | 8.0, 5.0) | |
| | | | 3.48 (ddd, 13.5, | |
| | | | 6.5, 4.5) | |
| 13 | — | 155.7 | — | 152.4 |
| OCH₃ | 4.10 (s) | 66.8 | 4.11 (s) | 66.7 |
| CH₃CO | — | — | — | 174.0 |
| CH₃CO | — | — | 2.21 (s) | 24.1 |

TABLE 3
¹H and ¹³C NMR data of Compound V and VI (CD₃OD, 500 and
125 MHz).

| | Compound V | | Compound VI | |
|---|---|---|---|---|
| N° | ¹H (Multiplicity, J) | ¹³C | ¹H (Multiplicity, J) | ¹³C |
| 2 | 7.60 (s) | 124.7 | 7.67 (s) | 125.0 |
| 3 | — | 112.3 | — | 111.5 |
| 4 | — | 123.5 | — | 123.2 |
| 5 | 7.99 (s) | 124.6 | 8.05 (s) | 124.5 |
| 6 | — | 119.3 | — | 119.5 |
| 7 | — | 116.4 | — | 116.6 |
| 8 | 7.85 (s) | 114.6 | 7.88 (s) | 114.6 |
| 9 | — | 133.6 | — | 133.6 |
| 10 | 4.94 (dd, 8.0, 5.0) | 47.4 | 5.13 (dd, 7.0, 5.0) | 47.6 |
| 11 | 2.26 (dddd, 14.0, 8.0, 8.0, | 28.3 | 2.26 (dddd, 13.5, | 26.8 |
| | 5.5) | | 8.0, 7.0, 6.5) | |
| | 2.18 (dddd, 14.0, 5.0, 5.0, | | 2.18 (dddd, 13.5, | |
| | 4.5) | | 5.0, 4.5, 4.5) | |
| 12 | 3.45 (ddd, 13.0, 8.0, 4.5) | 38.3 | 3.59 (ddd, 13.5, 8.0, | 38.1 |
| | 3.39 (ddd, 13.0, 5.5, 5.0) | | 4.5) | |
| | | | 3.49 (ddd, 13.5, 6.5, | |
| | | | 4.5) | |
| 13 | — | 155.7 | — | 152.3 |
| OCH₃ | 4.11 (s) | 67.0 | 4.13 (s) | 67.1 |
| CH₃CO | — | — | — | 174.0 |
| CH₃CO | — | — | 2.22 (s) | 24.1 |

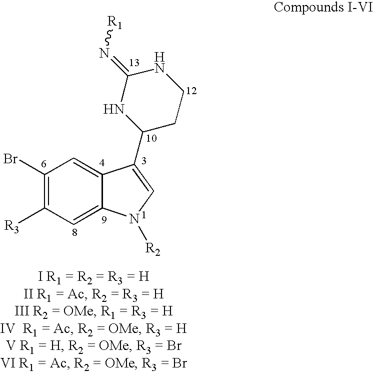

### Example 3

### Bioassays for Antitumor Screening

The finality of these assays is to interrupt the growth of an “in vitro” tumor cell culture by means of a continued exhibition of the cells to the sample to be testing.

### Cell Lines

| Name | N° ATCC | Species | Tissue | Characteristics |
|---|---|---|---|---|
| A549 | CCL-185 | human | lung | lung carcinoma “NSCL” |
| HT29 | HTB-38 | human | colon | colon adenocarcinoma |
| MDA-MB-231 | HTB-26 | human | breast | breast adenocarcinoma |

### Inhibition of Cell Growth by Colorimetric Assay

A calorimetric type of assay, using sulforhodamine B (SRB) reaction has been adapted for a quantitative measurement of cell growth and viability [following the technique described by Philip Skehan et al. (1990), New calorimetric cytotoxicity assay for anticancer drug screening, J. Natl. Cancer Inst., 82:1107-1112].

This form of assay employs 96 well cell culture microplates of 9 mm diameter (Faircloth et al. Methods in cell science, (1988), 11(4), 201-205; Mosmann et al, Journal of Immunological. Methods (1983), 65(1-2), 55-63). Most of the cell lines are obtained from American Type Culture Collection (ATCC) derived from different human cancer types.

Cells are maintained in RPMI 1640 10% FBS, supplemented with 0.1 g/L penicillin and 0.1 g/L streptomycin sulphate and then incubated at 37° C., 5% CO₂ and 98% humidity. For the experiments, cells were harvested from subconfluent cultures using trypsin and resuspended in fresh medium before plating.

Cells are seeded in 96 well microtiter plates, at 5×10³ cells per well in aliquots of 195 μL medium, and they are allowed to attach to the plate surface by growing in drug free medium for 18 hours. Afterward, samples are added in aliquots of 5 μL in a ranging from 10 to 10⁻⁸ μg/mL, dissolved in DMSO:EtOH:PBS (0.5:0.5:99). After 48 hours exposure, the antitumor effect is measured by the SRB methodology: cells arc fixed by adding 50 μL of cold 50% (wt/vol) trichloroacetic acid (TCA) and incubated for 60 minutes at 4° C. Plates are washed with deionised water and dried. 100 μL of SRB solution (0.4% wt/vol in 1% acetic acid) is added to each microtiter well and incubated for 10 minutes at room temperature. Unbound SRB is removed by washing with 1% acetic acid. Plates are air dried and bound stain is solubilized with Tris buffer. Optical densities are read on an automated spectrophotometric plate reader at a single wavelength of 490 nm.

The values for mean +/− SD of data from triplicate wells are calculated. Some parameters for cellular responses can be calculated: GI=growth inhibition, TGI=total growth inhibition (cytostatic effect) and LC=cell killing (cytotoxic effect).

### Antimitotic Assay Protocol

The mitotic ratio of cell culture was determined using a specific microplate immunoassay (ELISA). HeLa cells (h-cervix carcinoma, ATCC# CCL-2) were incubated in the presence or absence of the indicated compounds in 96 well microtiter plates After 18 hours, cells were washed with PBS and lysed on ice in 75 μL of freshly prepared lysis buffer (1 mM EGTA (pH 7.5), 0.5 mM PMSF and 1 mM NaVO₃) for 30 min. An aliquot of the cell extract (60 μL) was transferred to a high-binding surface ELISA plate and dried in a speed-vac for 2 h at room temperature. Plates were then blocked in 100 μL PBS-1% BSA for 30 min at 30° C. and sequentially incubated with anti-MPM2 primary mouse monoclonal antibody (Upstate Biotechnology, cat # 05-368) for 18 h at 4° C. and appropriate peroxidase-conjugated secondary antibody for 1 h at 30 ° C. After intensive washing in 0.02% Tween-20, peroxidase reaction was performed using 30 μL of TMB (3,3′,5,5′-tetramethyl-benzidine) for 30 min at 30° C. Reaction was stopped by adding 30 μL of a 4% H₂SO₄ solution. Assay was quantified by measuring the O.D. at 450 nm in a microplate spectrophotometer. Results were expressed as compound concentration that produces 50% of the control (taxol) mitotic ratio.

Tables 4-5 illustrates data on the biological activity of the compounds of the present invention.

TABLE 4
Cytotoxicity assay - Activity Data (Molar)

| | | Compound | Compound | Compound | Compound |
|---|---|---|---|---|---|
| | | I | IV | V | VI |
| MDA- | GI₅₀ | 4.18E−7 | 4.11E−7 | 7.96E−6 | 8.11E−7 |
| MB-231 | TGI | 1.52E−6 | 1.37E−6 | 2.06E−5 | 1.98E−6 |
| | LC₅₀ | 6.26E−6 | 5.20E−6 | n.d. | 4.95E−6 |
| HT29 | GI₅₀ | 3.88E−7 | 3.29E−7 | 7.96E−6 | 4.73E−7 |
| | TGI | 5.67E−7 | 4.65E−7 | 1.74E−5 | 9.23E−7 |
| | LC₅₀ | n.d. | 7.67E−7 | n.d. | 2.48E−6 |
| A549 | GI₅₀ | 6.56E−7 | 6.30E−7 | 8.70E−6 | 1.31E−6 |
| | TGI | n.d. | 7.12E−7 | n.d. | 4.05E−6 |
| | LC₅₀ | n.d. | n.d. | n.d. | 1.15E−6 |

n.d. = not determined

TABLE 5
Antimitotic assay - Activity Data (Molar)

| | IC₅₀ |
|---|---|
| Compound II | 1.19E−6 |
| Compound IV | 1.09E−6 |
| Compound VI | 1.80E−7-3.60E−8 |

1. A compound of general formula I: 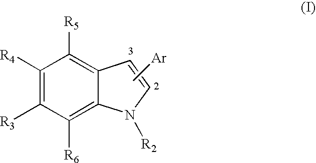
wherein Ar is an heterocyclic group of formula 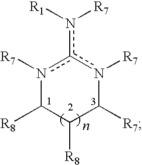
each R₁, R₂ and R₇ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, NRₐR_{b}, NRₐCOR_{b}, SO₂Rₐ, COORₐ, CORₐ, CONRₐR_{b}, ORₐ and OCORₐ;
each R₃, R₄, R₅, R₆ and R₈ is independently selected from hydrogen, substituted or unsubstituted CI-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, halogen, CN, NO₂, COORₐ, CORₐ, CONRₐR_{b}, ORₐ, OCORₐ, NRₐR_{b} and NRₐCOR_{b};
n is selected from 0 and 1;
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, and substituted or unsubstituted heterocyclic group; the Ar group may be attached to the carbon atoms 2 or 3 of the indole group through its atoms 1, 2 or 3;
the dotted lines represent one optional additional bond, with the proviso that when said additional bond exists the N atom bearing the double bond lacks the R₇ group; or
a pharmaceutically acceptable salt, derivative, tautomer, prodrug or stereoisomer thereof; with the exception of a compound of formula:

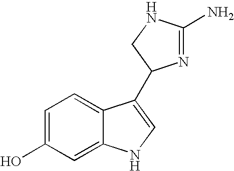

2. A compound according to claim 1, wherein n is 1.

3. A compound according to claim 1, having the following general formula II: 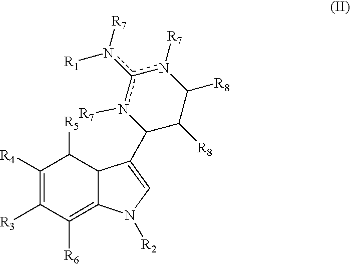
wherein R₁ to R₈ and the dotted lines are as defined in claim 1.

4. A compound according to any of claims 1 or 3, wherein R₁ and R₇ are independently selected from hydrogen, substituted or unsubstituted C₁-C₁₂ alkyl, substituted or unsubstituted aryl, ORₐ and CORₐ, and wherein Rₐ is as defined in claim 1.

5. A compound according to claims 1 or 3, wherein R₂ is selected from hydrogen, substituted or unsubstituted C₁-C₁₂ alkyl, substituted or unsubstituted aryl, ORₐ and CORₐ, and wherein Rₐ is as defined in claim 1.

6. A compound according to claims 1 or 3, wherein R₃, R₄, R₅ and R₆ are independently selected from hydrogen, halogen, ORₐ, OCORₐ, NRₐR_{b}, NRₐCOR_{b}, and wherein Rₐ and R_{b} are as defined in claim 1.

7. A compound according to claims 1 or 3, wherein R₈ is selected from hydrogen, halogen, NRₐR_{b} and NO₂, and wherein Rₐ and R_{b} are as defined in claim 1.

8. A compound according to claims 1 or 3, wherein one additional bond is present in one of the dotted lines.

9. A compound according to any of claims 1 or 2, having the following general formula III: 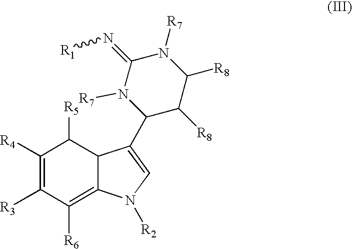
wherein R₁ to R₈ are as defined in claim 1 and the wavy bond ( [symbol] ) means that the double bond can exist as (E)-isomer or (Z)-isomer.

10. A compound according to claim 9, wherein R₁ is selected from hydrogen and CORₐ, wherein Rₐ is a substituted or unsubstituted C₁-C₆ alkyl.

11. A compound according to claim 9, wherein R₂ is selected from hydrogen and ORₐ, wherein Rₐ is a substituted or unsubstituted C₁-C₆ alkyl.

12. A compound according to claim 9, wherein R₃, R₄, R₅ and R₆ are independently selected from hydrogen and halogen.

13. A compound according to claim 12, wherein R₃, R₄, R₅ and R₆ are independently selected from hydrogen and Br.

14. A compound according to claim 9, wherein R₇ and R₈ are hydrogen.

15. A compound according to claim 9, having one of the following structures: 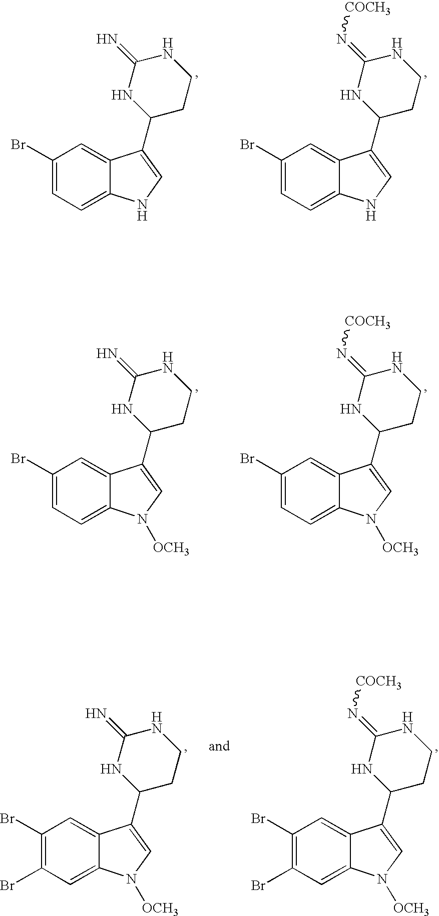
or a pharmaceutically acceptable salt, derivative, tautomer, prodrug or stereoisomer thereof.

16. A pharmaceutical composition comprising a compound of general formula I: 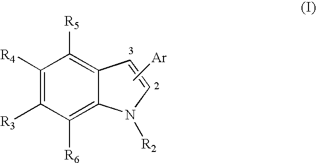
wherein Ar is an heterocyclic group of formula 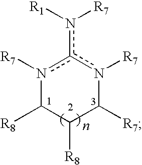
each R₁, R₂ and R₇ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, NRₐR_{b}, NRₐCOR_{b}, SO₂Rₐ, COORₐ, CORₐ, CONRₐR_{b}, ORₐ and OCORₐ;
each R₃, R₄, R₅, R₆ and R₈ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, halogen, CN, NO₂, COORₐ, CORₐ, CONRₐR_{b}, ORₐ, OCORₐ, NRₐR_{b} and NRₐCOR_{b};
n is selected from 0 and 1;
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, and substituted or unsubstituted heterocyclic group; the Ar group may be attached to the carbon atoms 2 or 3 of the indole group through its atoms 1, 2 or 3;
the dotted lines represent one optional additional bond, with the proviso that when said additional bond exists the N atom bearing the double bond lacks the R₇ group; or
a pharmaceutically acceptable salt, derivative, tautomer, prodrug or stereoisomer thereof;
and a pharmaceutically acceptable carrier or diluent.

17. A pharmaceutical composition according to claim 16, wherein the compound of formula I has the following general formula II: 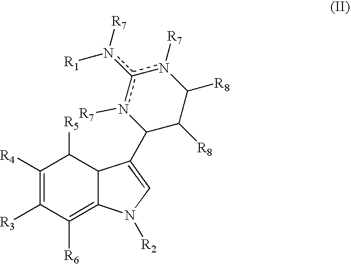
wherein R₁ to R₈ and the dotted lines are as defined in claim 16.

18. (canceled)

19. (canceled)

20. A method of treating any mammal, affected by cancer which comprises administering to the affected individual a therapeutically effective amount of a compound of general formula I: 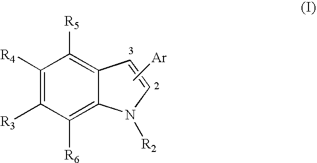
wherein Ar is an heterocyclic group of formula 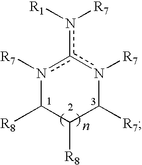
each R₁, R₂ and R₇ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, NRₐR_{b}, NRₐCOR_{b}, SO₂Rₐ, COORₐ, CORₐ, CONRₐR_{b}, ORₐ and OCORₐ;
each R₃, R₄, R₅, R₆ and R₈ is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, substituted or unsubstituted heterocyclic group, halogen, CN, NO₂, COORₐ, CORₐ, CONRₐR_{b}, ORₐ, OCORₐ, NRₐR_{b} and NRₐCOR_{b};
n is selected from 0 and 1;
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted arylalkyl, substituted or unsubstituted arylalkenyl, and substituted or unsubstituted heterocyclic group;
the Ar group may be attached to the carbon atoms 2 or 3 of the indole group through its atoms 1, 2 or 3;
the dotted lines represent one optional additional bond, with the proviso that when said additional bond exists the N atom bearing the double bond lacks the R₇ group; or
a pharmaceutically acceptable salt, derivative, tautomer, prodrug or stereoisomer thereof.

21. A method according to claim 20, wherein the compound of formula I has the following general formula II: 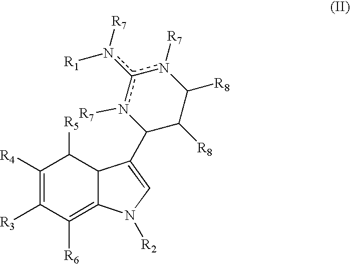
wherein R₁ to R₈ and the dotted lines are as defined in claim 20.

22. A process for obtaining a compound having one of the following structures: 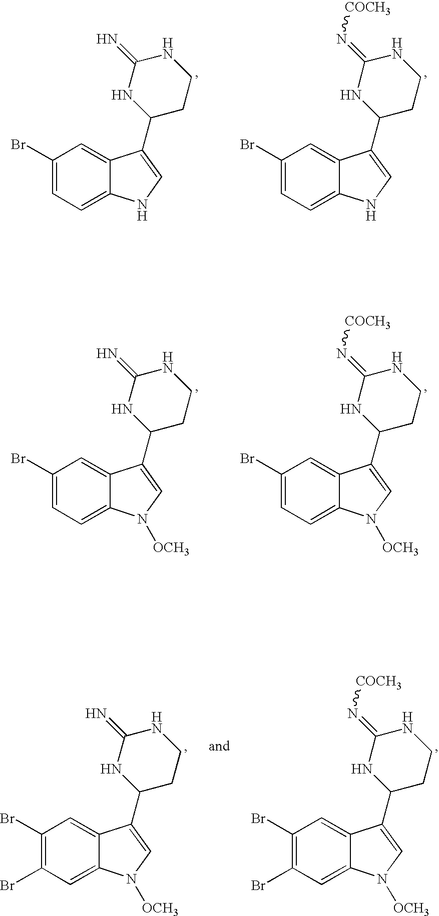
which comprises an extraction and isolation from Aplidium cyaneum.

23. The method of claim 20, wherein the affected individual is a human.
